(19) United States
(12) Patent Application Publication
(10) Pub. No.: US 2024/0374337 A1
(43) Pub. Date: Nov. 14, 2024

(54) TAMPER-EVIDENT CLOSURE WITH AN ACTUATABLE DOOR

(71) Applicant: 3M INNOVATIVE PROPERTIES COMPANY, St. Paul, MN (US)

(72) Inventors: Alan R. Dombrowski, Woodbury, MN (US); Adam S. Troness, Dellwood, MN (US); Val Vossen, Minneapolis, MN (US); Dennard J. Powell, St. Paul, MN (US); Kimberly A. Schommer, Hudson, WI (US)

(21) Appl. No.: 18/682,188

(22) PCT Filed: Jul. 29, 2022
(86) PCT No.: PCT/IB2022/057086

§ 371 Date: Feb. 8, 2024

Related U.S. Application Data
(60) Provisional application No. 63/232,892, filed on Aug. 13, 2021.

(51) Int. Cl. A61B 50/30; A61B 50/00; A61B 90/00; A61M 25/00; B65D 43/16; B65D 55/06

(52) U.S. Cl. CPC A61B 50/30; B65D 43/162; B65D 55/06; A61B 2050/0053; A61B 2050/0078; A61B 2090/081; A61M 25/002; B65D 2401/15

(57) ABSTRACT

The disclosed tamper-evident closure (100) encloses a medical access device (200), such as a vascular access device and includes an actuatable door (127) to create an optional access point (113) for accommodating various arrangements of medical access devices within the tamper-evident closure. The tamper-evident closure comprises a holding cavity (110), an access point into the holding cavity, an actuatable door covering the access point, and a hinge (127c) connecting the actuatable door to the holding cavity.

### TECHNICAL FIELD

The present disclosure relates to a tamper-evident closure. In particular, the present disclosure relates to a tamper-evident closure for medical access devices, such as intravenous access points, with an actuatable door.

### BACKGROUND

Patients receiving medical care will commonly have a medical access device, such as a vascular access device, inserted to assist the healthcare provider in delivering medications to the patient. Some medical access devices are for allowing fluids to be removed from a patient, like a urinary catheter or a surgical drain line. When a person is under the care of a healthcare provider it is important to control the medications that are delivered to the patient. Patients with psychiatric conditions, intellectually delayed patients, pediatric patients, or intravenous drug users may tamper with intravenous access points or intentionally deliver drugs or medications to the vascular access device. Unprescribed drugs entering the vascular access device can cause medical problems, drug interactions, blood stream infections.

### SUMMARY

The disclosed tamper-evident closure encloses a medical access device. The disclosed tamper-evident closure encloses a medical access device, such as a vascular access device and includes an actuatable door to create an optional access point for accommodating various arrangements of medical access devices within the tamper-evident closure. The tamper-evident closure comprises a holding cavity, an access point into the holding cavity, an actuatable door covering the access point, and a hinge connecting the actuatable door to the holding cavity.

Typically, for a tamper-evident closure, controlling the openings and access into the holding cavity limits the ability for a user to improperly access the medical access device. The tamper-evident closure can include additional features, as described, to make it visibly apparent that the tamper-evident closure has been modified if the medical access device contained in the tamper-evident closure becomes accessed. The tamper-evident closure cannot be reapplied over the medical access device in a way to look the same as it did prior to exposing the medical access device.

In one embodiment, the actuatable door has a releasable connection to the holding cavity on a first side and second side, and wherein the hinge connecting the actuatable door is between the first side and second side. In one embodiment, the actuatable door has a releasable connection to the holding cavity on a first side and bottom side, and wherein the hinge connecting the actuatable door is on a second side. In one embodiment, the hinge is a living hinge. In one embodiment, the hinge maintains connection of the actuatable door to the tamper-evident closure following actuation.

In one embodiment, the tamper-evident enclosure further comprises a door stop within the holding cavity to pressure fit with the actuatable door when the actuatable door is hingedly actuated into the holding cavity. In one embodiment, the door stop is a protrusion within the holding cavity and pressure fits with only a portion of the actuatable door when the actuatable door is hingedly actuated into the holding cavity.

In one embodiment, the tamper-evident closure further comprises a door support wall extending from the second part to engage with the actuatable door when the actuatable door is not actuated and is in connective engagement with the first part.

In one embodiment, the holding cavity further comprises reinforcing ribs extending along a direction of the actuation of the actuatable door. In one embodiment, the holding cavity comprises a first part and a second part forming the holding cavity.

In one embodiment, the tamper-evident closure further comprises a securing strap secured to the holding cavity. In one embodiment, the securing strap comprises a first securing strap portion secured to the first part, a second securing strap portion with a free end for removably securing to the second part, and an area of weakness separating the first securing strap portion from the second securing strap portion, wherein the force to break the area of weakness is less than the force to separate the securing strap from the first part or second part.

A method of enclosing a medical access device into the tamper-evident closure comprises actuating the actuatable door for an access point. The method further comprises friction fitting the actuatable door to the door stop. In one embodiment, the method of enclosing a medical access device into the tamper-evident closure comprising maintaining the actuatable door in connection with the holding cavity and closing the holding cavity to engage the door support wall with the actuatable door

### BRIEF DESCRIPTION OF DRAWINGS

FIG. 1 is a perspective view of one embodiment of a tamper-evident closure in an open position to expose the holding cavity and to receive a medical access device with the tamper-evident closure having an actuatable door covering an access point;

FIG. 2 is a top view of FIG. 1;

FIG. 3 is a perspective view of the tamper-evident closure of FIG. 1 in a closed position, with a securing strap retained and with a medical access device contained in the holding cavity and the actuatable door covering an access point;

FIG. 4 is a perspective view of the tamper-evident closure of FIG. 1 with the actuatable door hinged to provide the access point into the holding cavity;

FIG. 5 is a top view of the tamper-evident closure of FIG. 4;

FIG. 6 is a sectional view of the tamper-evident closure of FIG. 3 through the securing strap surrounding the holding cavity, with the medical access device removed;

FIG. 7 is a perspective view of the tamper-evident closure of FIG. 3 with the securing strap broke at the area of weakness to indicate tampering;

FIG. 8 is a perspective view of the tamper-evident closure of FIG. 3 where the tear strip has been released to open the tamper-evident closure and break the securing strap.

While the above-identified drawings and figures set forth embodiments of the invention, other embodiments are also contemplated, as noted in the discussion. In all cases, this disclosure presents the invention by way of representation and not limitation. Numerous other modifications and embodiments can be devised by those skilled in the art, which fall within the scope and spirit of this invention. The figures may not be drawn to scale.

### DETAILED DESCRIPTION

Various designs for tamper-evident closures are disclosed. The disclosed tamper-evident closure 100 comprises a holding cavity 110 and an actuatable door 127 to optionally create an access point 113 so that the tamper-evident closure 100 can accommodate a wider assortment of medical access devices 200.

To enable tamper evidence the tamper-evident closure further comprises a securing strap 170, and optional closure mechanism 160 such that upon tampering or removal of the securing strap 170 there is a visually apparent indication that securing strap 170 had broken and has been released. The disclosed tamper-evident closures are well suited for securing a medical access device 200 for delivering fluid to a patient, removing fluid from a patient or connecting to other devices through a tube 220. In some embodiment, caps 230, such as a 3M™ Curos™ Disinfecting Cap that contain disinfectant are secured over the medical access device 200.

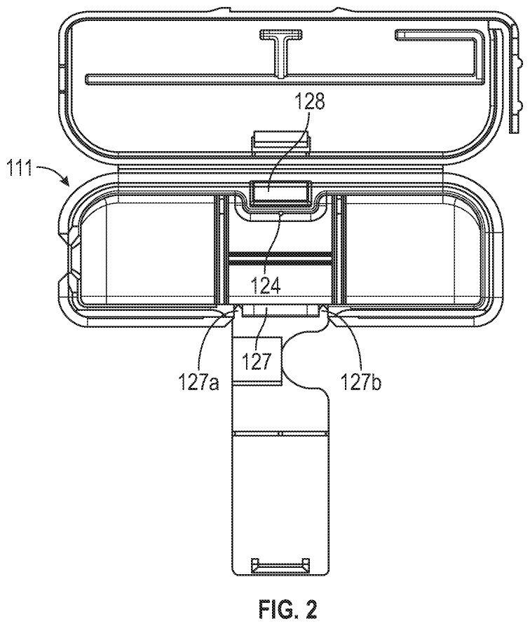

The holding cavity 110 has a length in the y direction, a width in the x direction, and a height in the z direction. The holding cavity has a perimeter 111 surrounding at least a portion of the holding cavity 110. At least one access point 113 is in the holding cavity 110, when the holding cavity is closed (FIG. 2, 5, 6) to allow for the tube 220 of the medical device to exit the holding cavity 110. Typically, the tamper evident closure 100 can be open so the access device 200 to be placed in the holding cavity 110 and closed to secure the access device 200 within the holding cavity 110.

An actuatable door 127 is in the holding cavity 110 and optionally provides for the access point 113. Some or all of the access points 113 could include actuatable doors 127. However, as shown in the embodiment, one access point 113 does not have an actuatable door, while an optional access point 113 in the side along the length of the tamper-evident closure 100 includes the actuatable door.

In one embodiment the securing strap 170 retains the tamper-evident closure 100 closed. In one embodiment, an optional closure mechanism 160 retains the tamper-evident closure 100 closed. Various design of the holding cavity 110, securing straps 170, and optional closure mechanism 160 are contemplated and disclosed herein.

The holding cavity 110 of the tamper-evident closure 100 contains the medical access device 200. The medical access device 200 can be any site on a catheter or intravenous tubing where medications can be administered and/or where fluid can be removed. The medical access device 200 can be a vascular access device that is a connection to the patient's vascular system for either administering medication or removing blood. The medical access device 200 can be any portion of the infusion tubing, catheter, tube, or other medical device. This can be any site on a catheter, intravenous tubing, or other topical or percutaneous medical device. This may be a luer active device (“LAD”) on a y-site, a LAD on the end of a line, or a male/female connection in the middle of the line or any other connection used on a topical or percutaneous medical device. A luer activated device can be either capped or un-capped.

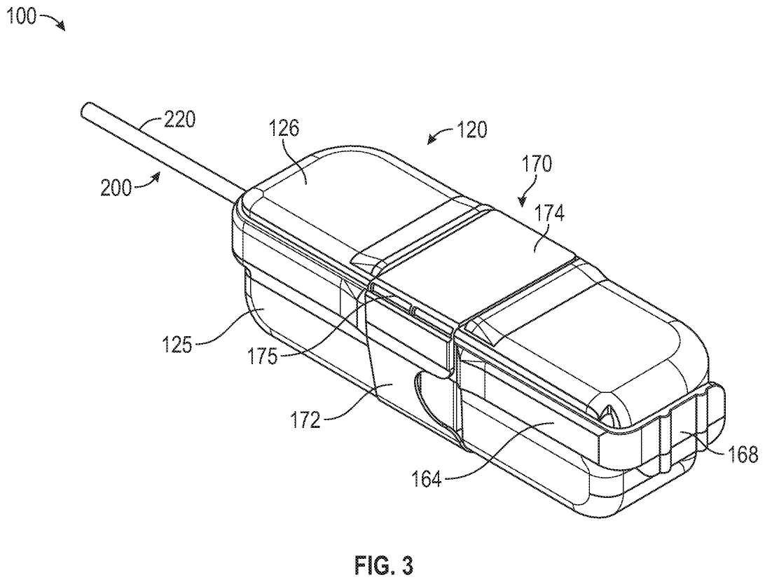

In some embodiments like shown in the figures, the fluid access point of the medical access device 200 is covered with a cap 230 (FIG. 3) for covering the fluid access point prior to use. Caps 230 can contain a disinfecting agent to keep the fluid access point 210 clean until use. Exemplary caps 230 are described in U.S. Pat. Nos. 7,282,186; 7,780,794; 9,907,617; 9,259,284, the disclosures of which are herein incorporated by reference. Exemplary caps 230 are sold as 3M™ CUROST™ Disinfecting Caps for Needleless Connectors, available from 3M Company.

When the holding cavity 110 is closed, the fluid access into the medical access device 200 is inaccessible to prevent access to the medical access device. If a use attempts to remove the securing strap 170 to open the tamper-evident closure, it will partially or completely separate and it will be visually apparent it has been tampered. If the tamper-evident closure 100 has been opened—either legitimately or through tampering—the securing strap 170 will completely separate and it will be visually apparent the tamper-evident closure 100 have been open. Further, it will not be possible to reclose the tamper-evident closure 100.

In the disclosed tamper-evident closure 100, the securing strap 170 comprises an area of weakness 175. The force to break the area of weakness 175 is less than the force to separate the securing strap 170 from the tamper-evident closure 100. Therefore, even without opening the holding cavity 110, tampering will be evident from the broken area of weakness 170.

In some embodiment, materials that are highly resistant to breaking by cutting or impact, such as plastics, films, metals or ceramics, could be used to make the tamper-evident closure a tamper-resistant or even tamper-proof closure.

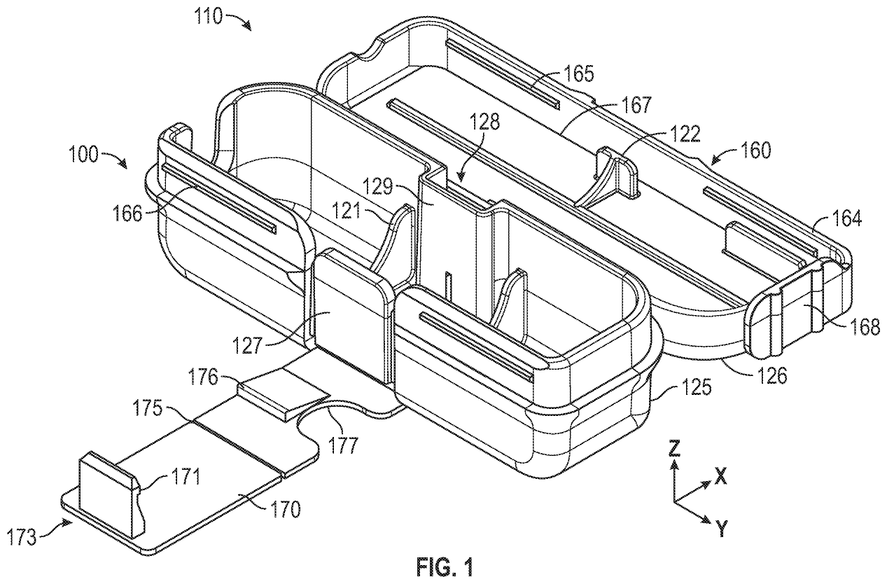

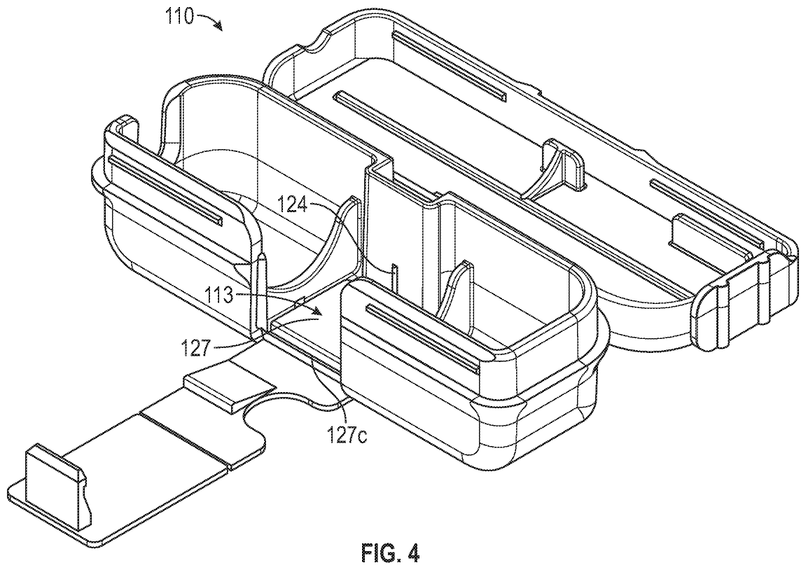

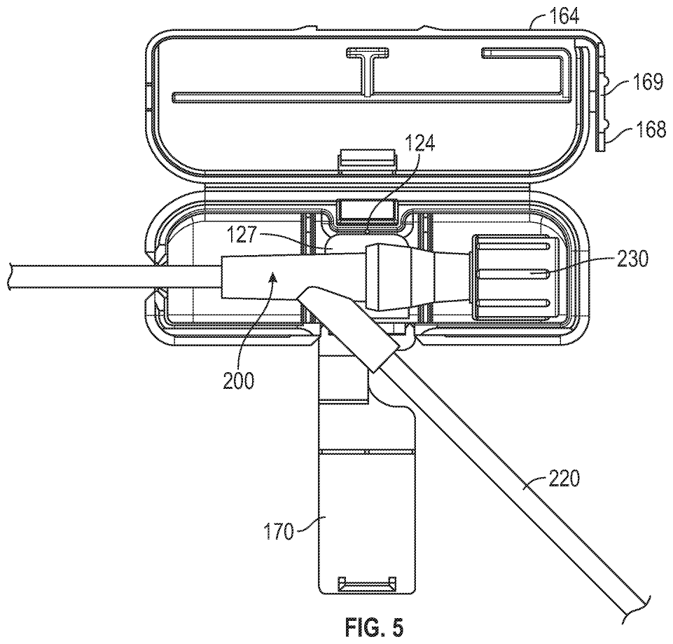

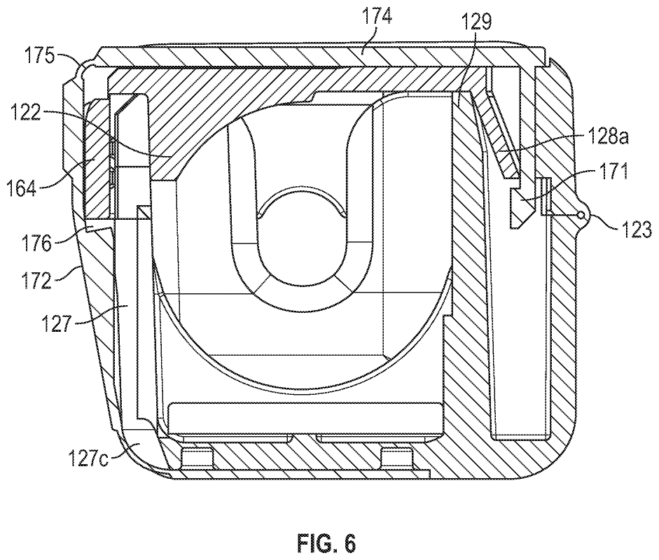

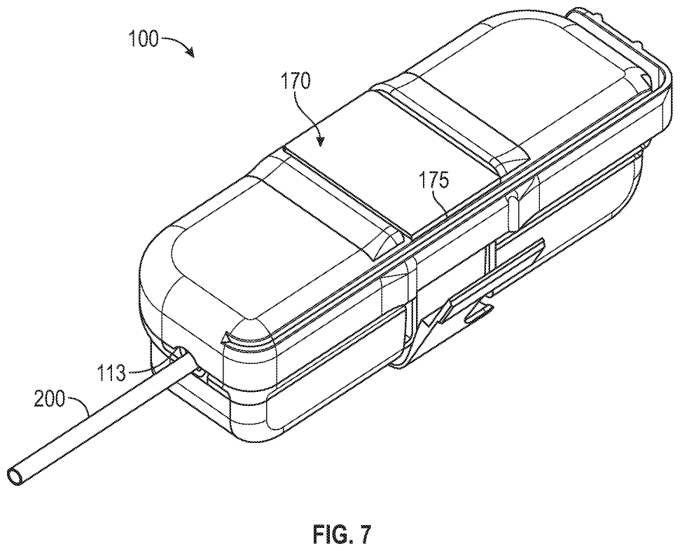

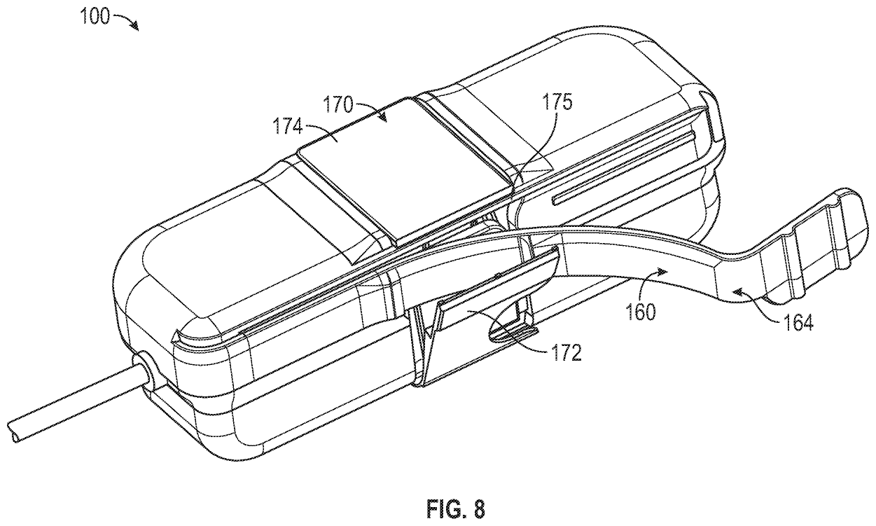

FIG. 1 is a perspective view of one embodiment of a tamper-evident closure 100 in an open position to expose the holding cavity 110 and to receive a medical access device within the tamper-evident closure 100, where there is an actuatable door 127 covering a potential access point. FIG. 2 is a top view of FIG. 1. FIG. 3 is a perspective view of the tamper-evident closure 100 of FIG. 1 in a closed position, with a securing strap 170 retained and with a medical access device 200 contained in the holding cavity 110 and the actuatable door 127 covering a potential access point. FIG. 4 is a perspective view of the tamper-evident closure 100 of FIG. 1 with the actuatable door 127 hinged to provide the access point 113 into the holding cavity 110. FIG. 5 is a top view of the tamper-evident closure of FIG. 4. FIG. 6 is a sectional view of the tamper-evident closure 100 of FIG. 3 through the securing strap 170 surrounding the holding cavity 110, with the medical access device removed. FIG. 7 is a perspective view of the tamper-evident closure 100 of FIG. 3 with the securing strap 170 broke at the area of weakness 175 to indicate tampering. FIG. 8 is a perspective view of the tamper-evident closure 100 of FIG. 3 where the tear strip 164 has been released to open the tamper-evident closure 100 and break the securing strap 170.

In this embodiment, the tamper-evident closure 100 comprises a first part 125 and a second part 126 that connect together to form the holding cavity 110. The medical access device 200 (FIGS. 2, 3, 5) can be placed in the first part 125, and then the second part 126 overlies the first part 125, like shown in FIG. 2. The first part 125 and second part 126 can be two separate parts or could be connected to one another by a hinge 123 (as best seen in FIG. 4). There are access points 113 in the tamper-evident closure 100 to allow the medical access devices tubing 220 to exit from the holding cavity 110 while the medical access device 200 is contained within the holding cavity 110. Other suitable designs of a tamper-evident closure 100 are shown and described in PCT Publication WO 2021/024139, the disclosure of which is here in incorporated by reference.

The securing strap 170 indicates tampering if a user was to attempt to remove the securing strap 170 from the tamper-evident closure 100 or to open the tamper-evident closure 100. In some embodiments, the securing strap 170 also functions to hold the tamper-evident closure 100 in a closed position. The securing strap 170 comprises a first securing strap portion 172 secured to the first part 125 and a second securing strap portion 174 that includes a free end 173 that is disconnected from the tamper-evident closure 100 before the tamper-evident closure 100 has been closed, like shown in FIGS. 1 and 3. In one embodiment, the first securing strap portion 172 is rigidly, integrally, or substantially permanently connected with the first part 125. Between the first securing strap portion 172 and the second securing strap portion 172 is an area of weakness 175 of the securing strap 170.

As shown in FIG. 3, once the first part 125 closes over the second part 126, the securing strap 170 extends from the first part 125 to the second part 126. In this embodiment, the securing strap 170 extends around a substantial portion of the tamper-evident closure 100. In this embodiment, the securing strap 170 extend from one side of the tamper-evident closure 100 to the opposing side of the tamper evident closure 100, across the entire height (z-direction) of the tamper-evident closure 100.

Various mechanism could be included for attachment of the second securing strap portion 174 to the tamper-evident closure 100. For example, mechanical of interlocking mechanism, adhesive attachments of one or both ends, etc. As shown in the embodiment, the second securing strap portion 174 to the second part 126. Typically, the attachment is a one-way, irreversible connection of the second securing strap portion 174. For example, in one embodiment, the mechanical attachment to allow for the free end 173 to secure to the second part 126 and then for the free end 173 to release from the second part 126 can be included. In the embodiment shown, the second securing strap portion 174 includes a latch extension 171 for mechanical engagement within a receiving latch 128a which is contained within a slot 128 (see FIGS. 3 and 4). The slot 128 passes through the second part 126 and within the slot 128 is the receiving latch 128a. In the embodiment shown, the latch extension 171 and receiving latch 128a interlock in a one-way, irreversible manner and therefore the interlock does not release. Here, the latch extension 171 will break if removal of the connection is attempted. This keeps the securing strap 170 securely connected to the first part 125 and second part 126. Also, in this embodiment, the securing strap 170 functions to hold the tamper-evident closure 100 in a closed position.

Optionally, within the holding cavity 110, the slot 128 for receiving the latch extension 171 is surrounded by a slot wall 129 that extends to prevent possible actuation of the latch extension 171 for release from the receiving latch 128a if tampering includes access from within the cavity pressing against the latch extension 171 (see FIG. 4). In other words, with an elongated tool, a user could press against the latch extension 171 and release it from the receiving latch 128a. The slot wall 129 is within the holding cavity 110. In the embodiment shown, the slot wall 129 extends from the first part 125 to the second part 126 and substantially surround the latch extension 171.

The latch extension 171 in this embodiment is a barb, but any number of other one-way mechanical interlocks are suitable. For example, the latch extension could be a protruding portion like a ball that inserts into a receiver.

The securing strap 170 includes an area of weakness 175 that enables controlled breaking when attempting to release the securing strap 170 or attempting to open the holding cavity 110. The area of weakness 175 could be any number of designs where the force to break the area of weakness 175 is less than the force to separate the securing strap 170 from the first part 125 or second part 126. In other words, attempted removal, opening, destruction of the securing strap 170 will break at the area of weakness 175 before the securing strap 170 connection of the latch extension 171 and slot 128 will break or release. For example, the area of weakness 175 can be a substantially thinner material than the other portions of the securing strap 170 or can be one or more perforations through the securing strap 170.

The slot 128 is shaped to be a container so that if the latch extension 171 broke from tampering, the latch extension 171 will separate from the free end 173 of the securing strap 170 and fall into the slot 128 and advantageously collects and contains the loose part of the broken latch extension 171.

FIG. 7 depicts the tamper-evident closure 100 in a closed position but where a user has attempted to lift, remove, or release the second securing strap portion 172 from attachment with the second part 126. Here, the area of weakness 175 has separated the first securing strap portion 172 from the second securing strap portion 174 to indicate tampering. The area of weakness 175 broke before the latch extension 171 broke or released from the slot 128.

In some embodiment, the first part 125 and the second part 126 are held in a close position by an additional closure mechanism 160. In this embodiment, a closure mechanism 160 for holding the holding cavity 110 closed comprises a tear strip 164 extends around at least a portion of the perimeter of the tamper-evident closure 100 connecting the first part 125 and second part 126. The tear strip 164 comprises a mechanical interlocking system linking between the first part 125 and the second part 126. In the embodiment shown, the tear strip 164 comprises a longitudinally extending protruding portion 165 that interlocks with a longitudinally extending receiving portion 166 on the first part 125 for securing the tamper-evident closure 100 in a closed position. It is understood that the extending or receiving portions could be alternative be included on the other first part 126 or second part 126. The tear strip 164 has a line of weakness 167 adjacent to the tear strip 164 that connects the tear strip 164 to the second part 126 in this embodiment, but it is understood is could alternatively connect the tear strip 164 to the first part 125.

The tear strip 164 can have a tab 168 for a user to be able to easily grab the tear strip 164 to begin removal of the tear strip 164. Removal of the tear strip 164 separates the tear strip 164 from the second part 126 along the line of weakness 167. The tab 168 could be anywhere along the tear strip 164. Typically, the tab 168 is on an end of the tamper-evident closure 100 without an access point 113 so as not to interfere with the tube 220 that leaves the access point 113. However, if the tab 168 is on an end also containing an access point 113, the tab 168 is dimensioned and positioned to not interfere with the exiting tube 220. The tab 168 is therefore ideally positioned slightly removed from the tamper-evident closure 100, like shown in the top view of FIG. 5. However, to ensure the tab 168 is not so disconnected and to help strengthen the connection of the tear strip 164 to the closed holding cavity 110, optional tab ties 169 are included to secure a portion of the tab 168 to the tamper-evident closure 100. These tab ties 169 add strength to the tab 168 connection to prevent the tab from snagging and inadvertently separating from the tamper-evident closure 100. Also, the tab 168 being separated from the tamper-evident closure 100 creates a slight area of weakness and the tab ties 169 secure the tab 168 adding strength to the closure.

In this embodiment with the tear strip 164, the securing strap 170 overlies the tear strip 164. To remove the tear strip 164, the tab 168 is pulled to break the line of weakness 167 that connects the tear strip 164 to the second part 126, and then further, the tear strip 164 breaks the area of weakness 175 in the securing strap 170, as best shown in FIG. 8. Removal of the tear strip 164 removes the longitudinally extending protruding portion 165 to disengage the longitudinally extending protruding portion 165 from the longitudinally extending receiving portion 166. Therefore, when the tear strip 164 is used to open the tamper-evident closure 100, it disengages the locking connection between the first part 125 and the second part 126. Further, the tear strip 164 breaks the area of weakness 175 of the securing strap 170 irrevocably breaks the securing strap 170 so tampering is evident without the need for a secondary tool.

It is understood the closure mechanism 160 could accomplish the same function in other ways, such as, for example, the second part 126 could include the longitudinally extending protruding portion 165 and the first part 125 could include the longitudinally extending receiving portion 166 that interlocks with the longitudinally extending protruding portion 165. Further alternative closure mechanism 160 could be used to secure the holding cavity closed, such as those described in PCT Publication WO 2021/024139, the disclosure of which is here in incorporated by reference.

In this embodiment with the securing strap 170 overlying a tear strip 164, the securing strap 170 can include a ramp edge 176, which align with the tear strip 164 (see FIG. 6). As the tear strip 164 is removed, the ramp edge 176 maintains alignment of the tear strip 164 with the area of weakness 175 so that the force applied by the tear strip 164 to the area of weakness 175 easily breaks the area of weakness 175. Without the ramp edge 176, the tear strip 164 might slide along the securing strap 170 and be misaligned with the area of weakness 175.

The tamper-evident closure 100 can include various access points 113. However, access points 113 can introduce points of egress into the holding cavity 110. For example, the tamper-evident closure 100 could include access points 113 on both opposing ends of the tamper-evident closure. But if an access point 113 is provided on both ends, then the medical access device 200 will include a cap 230 to prevent access into the medical access device 200. For accommodating various medical access devices 200, the tamper-evident closure 100 has an actuatable door 127 is in the holding cavity 110 for creating an access point 113. As shown in FIG. 3, the medical access device 200 only has a single tube 220, and in this embodiment the actuatable door 127 remains closed. As shown in FIG. 5, the medical access device 200 is a Y-site device with a side tube 220 and therefore the actuatable door 127 is pressed inward into the holding cavity 110.

The actuatable door 127 covering the potential access point is shown in FIG. 1. The actuatable door 127 has a hinge 127c that connects the actuatable door 127 to the holding cavity 110. Following actuation, the hinge 172c retains the connection between the actuatable door 127 and the tamper-evident closure 100. In this embodiment, the hinge 127c is a living hinge of the same material as the surrounding holding cavity 110, and best seen in FIG. 6. This is desirable because even upon actuation, the actuatable door 127 does not separate from the tamper-evident closure 110. Instead, the actuatable door 127 remains connected. Other hinge designs could be including, including a pin though openings. The hinge 127c is shown on the bottom, but the hinge 127c could be at one of the side walls 127a or 127b. Having the hinge 127c retain the actuatable door 127 to the tamper evident closure 100 is desirable because loose pieces and fragments of the tamper-evident closure 100 would be undesirable in a health care setting for possible choking hazard, for clean-up, and for inadvertent introduction into a surgical field.

To fully secure the actuatable door 127 into the holding cavity, the holding cavity 110 has a door stop 124. The door stop 124 pressure fits with the actuatable door 127 when the actuatable door 127 hingedly actuated into the holding cavity 110. In the embodiment shown best in FIG. 2, the door stop 124 is a protrusion within the holding cavity opposite from the actuatable door 127 and pressure fits with only a portion of the actuatable door when the actuatable door is hingedly actuated into the holding cavity.

The portions of the actuatable door 127 without the hinge 127c can be held to the holding cavity 110 such as shown on a first side 127a and second side 127b. First side 127a and second side 127b are typically structurally weak areas, such as a thin wall as shown in FIG. 2, or with perforations, to allow for easy disconnection of the actuatable door 127 at these areas. A continuous thin wall such as shown best in FIG. 2 is more desirable than perforations because perforations create small openings that are areas of egress into the holding cavity 110. The thin portions of 127a and 127b recess from the exterior of the tamper-evident closure 100, like shown in FIG. 2 or could be substantially planar on the exterior with the thin portions on the interior of the holding cavity 110.

The second part 127 can include a door support wall 122. When the actuatable door 127 is not actuated, but left closing the potential access point (like FIGS. 3 and 6), then the actuatable door 127 abuts the door support wall 122 preventing the actuatable door 127 for pressing inward and allowing egress into the holding cavity 110.

The tamper-evident closure 100 can include reinforcing ribs 121 to add structural strength to the holding cavity. As described, typically the tamper-evident closure 100 is length is greater than the width, this could cause weakness along the mid-section of the length. That weakness could allow a user to slightly squeeze and deform the tamper-evident closure 100. The deformation could disconnect the closure mechanism 160. The pressure to actuate the door 127 might cause the tamper-evident closure 100 to deform or flex too much making it difficult to actuate the door 127. Or if an actuatable door 127 is included, slight deformation might increase the space in and around the door 127 to all a user to insert elongated objects into the holding cavity 110.

As shown in this embodiment, the securing strap 170 can overlay this removable door 127 and if it overlies the removable door 127, then the securing strap 170 includes a partial opening to allow for the medical access device 200 to exit the tamper-evident closure 100 and therefore accommodate Y-site medical access devices 200.

The securing strap 170 in this embodiment overlies the optional actuatable door 127. To allow for a potential tube 220 to exit from the access point 113 created when the actuatable door 127 is lowered, the securing strap 170 has a notch 177 through a portion aligned with the actuatable door 127.

In some embodiments, the securing strap 170 itself could include at the notch 177 the actuatable door 127 that is filled in and covers the access point 113, or if removed provides an access point 113 for a tube 220 into the holding cavity.

In alternative designs of the tamper-evident closure containing a securing strap 170, the securing strap 170 itself serves as the primary closure holding the first part 125 together with the second part 126.

In such an embodiment, the area of weakness 175 separates and then allow for the first part 125 and second part 126 to open and allow access into the holding cavity 110. In this type of construction the securing strap 170 could include a tab 168 to allow for easy, intentional separation of the first securing strap portion 172 from the second securing strap portion 175.

It is understood that both the first securing strap portion 172 and the second securing strap portion 174 could include one-way engaging fasteners such as described herein with the latch extension 171 and slot 128. It is understood that although the description of the securing strap 170 is with the second securing strap portion 174 securing to the second part 126, instead the securing strap 170 might instead have the first securing strap portion 172 securing to the first part 125 as described herein.

The securing strap 170 is shown aligned over the actuatable door 127. In other embodiments, the securing strap 170 can be along any portion of the tamper-evident closure 100, where the first securing strap portion 172 secures with the first part 125 and the second securing strap portion 174 secures to the second part. Typically, the securing strap 170 will span the entire height (z-direction) of the tamper evident closure 100.

In use, when desiring to use the access point, the actuatable door 127 is pressed inward to the holding cavity 110. When there is a door stop 124, the actuatable door 127 is friction fit and pressed against the door stop 124 to secure the actuatable door 127 in place, such as shown in FIG. 5. Then, the medical access device 200 is placed in the open tamper-evident closure 100 such as shown in FIG. 5. The first part 125 and second part 126 close to form the holding cavity 110 around the medical access device 200. The free end 173 of the securing strap 170 is engaged with the second part 126 to hold the first part 125 and second part 126 closed. Specifically, the latch 171 locks in the slot 128. If included, the underlying closure mechanism 160, a tear strip 164 as shown, secures the first part 125 and second part 126 closed. In this embodiment the tear strip 164 aligns generally with the area of weakness 175 of the securing strip 170. The medical access device 200 is contained in the holding cavity 110 and access to the medical access device 200 is very limited.

To open the tamper-evident closure 100, the securing strap 170 is broken, which may separate the first part 125 from the second part 126. When there is a closure mechanism 160, such as the tear strip 164 shown, the tab 168 on the tear strip 164 is pulled to release the first part 125 from the second part 126, and the tear strip 164 also breaks the area of weakness 175 to separate the first securing strap portion 172 from the second securing strap portion 174.

To further aid in seeing any visual tampering, it could be that the securing strap 170 is one color and the tamper-evident closure 100 is a different color. Tamper-evident closures, such as those disclosed in this application, are designed to be closed and opened without the need for additional removal tools. For example, perforation lines, breakable securement straps, twistable or tearable structures or film can be used so that when a health care provider intentionally wants to access the vascular access device, it is easy to remove the tamper-evident closure. Also, it is desirable that when the tamper-evident closure is intentionally removed that the holding cavity opens cleanly with few separating parts.

The tamper-evident closure achieves the benefit of providing a strong holding cavity with limited ingress access into the holding cavity, that is easy to open without extra tools, and easy to break the securing strap while also being modular to accommodate medical devices with varying numbers of tubes.

Although specific embodiments have been shown and described herein, it is understood that these embodiments are merely illustrative of the many possible specific arrangements that can be devised in application of the principles of the invention. Numerous and varied other arrangements can be devised in accordance with these principles by those of skill in the art without departing from the spirit and scope of the invention. The scope of the present invention should not be limited to the structures described in this application, but only by the structures described by the language of the claims and the equivalents of those structures.

1. A tamper-evident closure for a medical access device comprising:
a holding cavity;
at least one access point into the holding cavity;
an actuatable door covering the access point
a hinge connecting the actuatable door to the holding cavity.

2. The tamper-evident closure of claim 1, wherein the actuatable door has a tearable connection to the holding cavity on a first side and second side, and wherein the hinge connecting the actuatable door is on a third side between the first side and second side.

3. The tamper-evident closure of claim 1, wherein the actuatable door has a tearable connection to the holding cavity on a first side and third side, and wherein the hinge connecting the actuatable door is on a second side.

4. The tamper-evident closure of claim 1, wherein the hinge is a living hinge.

5. The tamper-evident closure of claim 1, wherein the hinge maintains connection of the actuatable door to the tamper-evident closure following actuation.

6. The tamper-evident closure of claim 1, further comprising a door stop within the holding cavity to pressure fit with the actuatable door when the actuatable door is hingedly actuated into the holding cavity.

7. The tamper-evident closure of claim 6, wherein the door stop is a protrusion within the holding cavity and pressure fits with only a portion of the actuatable door when the actuatable door is hingedly actuated into the holding cavity.

8. The tamper-evident closure of claim 1, further comprising a door support wall extending from the second part to engage with the actuatable door when the actuatable door is not actuated and is in connective engagement with the first part.

9. The tamper-evident closure of claim 1, wherein the holding cavity further comprises reinforcing ribs extending along a direction of the actuation of the actuatable door and proximate the actuatable door.

10. The tamper-evident closure of claim 1, wherein the holding cavity comprises a first part and a second part forming the holding cavity.

11. The tamper-evident closure of claim 1, further comprising a securing strap secured to the holding cavity.

12. The tamper-evident closure of claim 11, wherein the securing strap comprises a first securing strap portion secured to the first part, a second securing strap portion with a free end for irreversibly securing to the second part, and an area of weakness separating the first securing strap portion from the second securing strap portion, wherein the force to break the area of weakness is less than the force to separate the securing strap from the first part or second part.

13. A method of enclosing a medical access device into the tamper-evident closure of claim 1, comprising:
actuating the actuatable door to form an access point.

14. The method of claim 13, further comprising:
friction fitting the actuatable door to the door stop.

15. A method of enclosing a medical access device into the tamper-evident closure of claim 8, comprising:
maintaining the actuatable door in connection with the holding cavity;
closing the holding cavity to engage the door support wall with the actuatable door.
